(19) United States
(12) Patent Application Publication
(10) Pub. No.: US 2006/0259329 A1
(43) Pub. Date: Nov. 16, 2006

(54) System and Method for Determining the Degree of Abnormality of a Patient's Vital Signs

(75) Inventor: Jeffrey A. Kline, Charlotte, NC (US)

Correspondence Address: BOND, SCHOENECK & KING, PLLC, ONE LINCOLN CENTER, SYRACUSE, NY, 13202-1355, US

(73) Assignee: CHARLOTTE-MECKLENBURG HOSPITAL AUTHORITY D/B/A CAROLINAS MEDICAL CENTER, Charlotte, NC (US)

(21) Appl. No.: 11/164,442

(22) Filed: Nov. 22, 2005

Related U.S. Application Data
(63) Continuation-in-part of application No. 10/267,134, filed on Oct. 8, 2002.
(60) Provisional application No. 60/371,284, filed on Apr. 9, 2002.

(51) Int. Cl. G06F 19/00; A61B 5/00

(52) U.S. Cl. 705/3; 128/920

(57) ABSTRACT

A system and method for determining the degree of abnormality of a vital sign of a patient by obtaining the clinical profile of said patient and determining the statistical difference between the vital sign of the patient and the vital signs of previously evaluated patients having similar clinical profiles. The vital signs of previously evaluated patients having similar clinical profiles are determined based on matching the attributes of the patent's clinical profile to the clinical profiles of previously evaluated patients. The statistical difference, and the patent's clinical profile may be exported to an electronic medical record system or printed in hard copy for inclusion in the patient's medial file.

### CROSS REFERENCE TO RELATED APPLICATION

The present application is a continuation-in-part of U.S. patent application Ser. No. 10/267,134, filed Oct. 8, 2002, which claims priority to U.S. Provisional Application Ser. No. 60/371,284, filed Apr. 9, 2002.

### BACKGROUND OF THE INVENTION

The present invention generally relates to a system and method for evaluating potentially fatal diseases and, more particularly, for determining the degree of abnormality of one or more vital signs of a patient as compares to previously evaluated patients having similar clinical profiles.

### DESCRIPTION OF PRIOR ART

As technology produces more rapid methods of evaluating a patient's risk for contracting a life threatening disease, physicians will avail themselves of these technologies more often, leading to an increase in resource use. Owing to new research in imaging and laboratory testing over the past five years, protocols for testing patients with complaints suggestive of the possibility of a life threatening illness in the emergency department have changed substantially at many centers. Three examples are the probability of pulmonary embolism (PE) in a patient with chest pain and/or shortness of breath, the probability of an acute coronary syndrome in a patient with anterior chest pain, and the probability of subarachnoid hemorrhage in a patient with a headache. For these conditions, emergency physicians are becoming more reliant on the use of specific tests and diagnostic protocols. For patients with possible PE, physicians can order a D-dimer assay and contrast-enhanced computerized tomography (CT). To rule out acute coronary syndrome, physicians can invoke a diagnostic protocol that includes serial blood chemistry studies and cardiac imaging. Finally, to rule out subarachnoid hemorrhage, physicians can perform CT scanning followed by lumbar puncture for evaluation of the cerbrospinal fluid. The problem with each of these examples includes increased time and cost required to complete the evaluation, and the possibility of false positive testing that can lead to more invasive and potentially more dangerous diagnostic studies and false positive diagnoses. The probability of adverse events related to false positive testing will increase in proportion to the frequency with which patients with a very pretest low probability are evaluated for these diseases.

Under the pressure of constant overcrowding and medical/legal concerns, emergency physicians are ready to embrace more rapid and streamlined systems to screen for a common and potentially fatal disease. At the same time, emergency medicine physicians are taught during residency, in the textbooks, and in continuing medical education courses, that they must have an unwavering suspicion for the potential that every patient with chest pain, shortness of breath or headache may have an undiagnosed fatal disease process, including acute coronary syndrome, pulmonary embolism (PE) and subarachnoid hemorrhage, respectively. As a result, many physicians working in the emergency department setting maintain the position that the liberal use of screening tests is ethically and medically/legally warranted.

As a result of these influences, the frequency of objective screening for acute coronary syndromes, PE, and subarachnoid hemorrhage has increased sharply, even as U.S. emergency departments become even more overcrowded. In 1998, when the scintillation ventilation-perfusion (VQ) lung scanning was the primary mode of evaluating PE, 0.39 percent of 96,000 emergency department patients underwent a VQ scan. However, in 2000, after implementation of CT scanning as the primary method of evaluation for PE, CT scans were performed to evaluate for PE in 0.69 percent of 102,000 emergency department patients. When the PI implemented a “rapid PE rule out” system in 2001 (consisting of a decision rule plus a whole-blood D-dimer plus an alveolar deadspace measurement) the rate of screening for PE increased to 1.4% of 108,000 patients.

When physicians in Canada used a scoring system and D-dimer as the first step to screen for PE in 946 ED patients, the resulting overall probability of PE in the study was reduced to 9.5 percent (the lowest yet reported), suggesting very liberal use of testing. Increased screening for PE may have negative consequences. A study by Goldstein et al, demonstrated that the implementation of a rapid D-dimer method to screen for PE produced a net increase in the rate of VQ scanning among inpatients.

These findings show that as technology produces more rapid and easier methods of evaluating for PE, that physicians will avail themselves of these technologies more often, potentially leading to an increase in resource use. As the frequency of screening for PE increases in relatively low-risk groups, the number of adverse events related to contrast allergy, radiation exposure, and anticoagulant treatment of false positive cases may increase. In other words, more rapid tests offer the option of easier evaluation for life-threatening illness, but at the risk of being overused in an extremely low-risk population. Moreover, as the rate and breadth of screening for potentially fatal disease increases in relatively low-risk groups, the number of adverse events related to contrast allergy, radiation exposure, and treatment of false positive cases will also increase.

The diagnostic accuracy of the objective tests, such as a computed tomography x-ray of the chest, can be defined by their likelihood ratio. Likelihood ratios are relatively precise variables that are arithmetically defined from sensitivity and specificity data provided by clinical studies. Moreover, meta-analysis techniques allow the aggregation of the results of many separate studies of one test, to estimate a composite likelihood ratio negative for the diagnostic test. However, no method exists to calculate a relatively precise estimate of the pretest probability of life-threatening diseases.

Traditional methods of pretest determination of life-threatening diseases involve a particular physician's remembered cases, the use of practice databases, planned research, and population prevalence. Although remembered cases offers an immediate and constantly available method, this “gestalt” method lacks reproducibility and is likely to vary with training level and can be subject to bias. Practice databases and population prevalence may be helpful for a gross estimate for a patient based upon one or two symptoms, but current strategies lack the ability to provide specialized consideration of age, gender, race, vital sign data, and the mosaic of clinical data for any given patient. The bulk of published methods for pretest assessment fall into the area of planned research. Multiple schemes and scoring systems have been devised to estimate the pretest probability of life-threatening diseases, including neural network systems scoring systems and various criteria based upon analysis of clinical factors with Boolean operators. These systems are logically designed and are relatively straight-forward to use. The drawback to existing methods of pretest assessment is that they either underfit or overfit individual patients, and only provide ranges of probability when, within each range, there exist domains of significantly different probabilities. For example, published scoring systems targeted at PE categorize up to 50 percent of ED patients as moderate risk, providing the vague assurance that the pretest probability lies between 20 to 60 percent. Published scoring systems are also hindered by their assumption that each variable functions independently to predict the presence or absence of disease of interest, and do not allow for a tailor-made clinical profile to be developed for every patient. As a result, patients with factors that represent a true risk for PE are overlooked in the derivation of the scoring system. Additionally, these methods do not factor the complex interdependence of predictors on the probability of the disease.

In the hospital and clinic setting, physicians and risk managers often wish to identify the risk of a particular vital sign or clinical feature. This concern also frequently arises in the case of civil litigation involving an accusation of negligence against a physician. For example, a physician may not evaluate a patient for an abnormal vital sign, such as a systolic blood pressure (BP) of 92 mm Hg. Under certain circumstances, a systolic BP of 92 mm Hg may be considered within normal limits. For example, it is frequently believed that females of small habitus will have a lower BP than a large male. Accordingly, this may compel a physician to ignore as systolic BP of 92 mm Hg and neither treat it with fluid infusion or perform any diagnostic studies, believing that this is within normal range for that individual.

Another example might be if a physician notices a pulse oximetry reading of 92% in a 72 year old smoker, the physician may believe that this low pulse oximetry reading (which is clearing abnormal compared with healthy subjects) is reasonably explained by the patient's age and previous lung injury from smoking. If a physician fails to take diagnostic action on these abnormalities, and an adverse outcome occurs, the issue of whether the physician deviated from standard care is often contentious. No existing method or system can determine the degree of abnormality for these patients compared with “like” or similar subjects, as defined by shared clinical characteristics such as age, gender, prior disease status.

The only conventional method of determining this normality is to ask for the experience of previous doctors, and to evaluate statistical summary data from published research of populations of patients that may share one trait with the patient of interest. The disadvantage of this method is that it is not possible to take the pages of a published study of, for example, 1,000 young women who participated in a birth control study, and parse out only the patients who are very similar to the small habitus female (in terms of age, gender, and body size), or to examine a study of 1,000 smokers and select out only males 72 years of age and determine their pulse oximeter readings.

### OBJECTS AND ADVANTAGES

It is a principle object and advantage of the present invention to provide physicians with an accurate method of evaluating a patient for the probability of the presence of a potentially life-threatening disease.

It is a further object and advantage of the present invention to provide a method for determining the probability of certain outcomes of a potentially life-threatening disease, including degree of severity of the disease and the probability of death within a defined interval.

It is an additional object and advantage of the present invention to provide a method of evaluating a patient for the probability of the presence of a potentially life-threatening disease which reduces the likelihood of unnecessary diagnostic testing.

It is a further object and advantage of the present invention to provide physicians with a method for evaluating a patient for the probability of the presence of a potentially life-threatening disease which incorporates numerous clinical factors that can be obtained by routine clinical interview and physical examination.

It is an additional object and advantage of the present invention to reduce the number of incorrect diagnoses.

It is a further object and advantage of the present invention to determine the probability of certain adverse outcomes which mandate emergent treatment or intervention.

It is an additional object and advantage of the present invention to improve the documentation of cases histories to reduce or eliminate associated malpractice issues.

It is also an object and advantage of the present invention to provide a system and method for identifying the risk associated with a particular vital sign or clinical feature.

Other objects and advantages of the present invention will in part be obvious and in part appear hereinafter.

### SUMMARY OF THE INVENTION

In accordance with the foregoing objects and advantages, the present invention provides a system and method for determining the degree of abnormality of at least one vital sign of a patient. First, the clinical profile of the patient, including at least one patent attribute and at least one patient vital sign of interest to be evaluated is obtained from the patient. Next, the clinical profile, including the attribute(s) and vital sign(s) are input into a data processing unit, such as a computer of personal digital assistant. The clinical profile of the patient is compared to the clinical profiles of previously evaluated patients to determine and retrieve the clinical profiles of previously evaluated patients that have attributes corresponding to current patent. The statistical difference or differences between the vital sign(s) of the patient and the vital signs of the previously evaluated patients may then be calculated. The results, including the patient's clinical profile, may be exported to an EMR system, printed for inclusion in the patient's medical chart, displayed for consideration by a physician, or stored in electronic format for future evaluation.

### BRIEF DESCRIPTION OF THE DRAWINGS

The present invention will be more fully understood and appreciated by reading the following Detailed Description in conjunction with the accompanying drawings, in which:

FIG. 1 is a flowchart of the method of the present invention.

FIG. 2 is an example of an electronic data form for use with the present invention.

FIG. 3 is a flowchart of a hypothetical determination of pretest probability according to the present invention.

FIG. 4 is a flowchart of an additional embodiment of the present invention.

FIG. 5 is a flowchart of a further embodiment of the present invention.

### DETAILED DESCRIPTION

Although this description refers to a pulmonary embolism (PE) as the primary disease, the method and system of the present invention may be used to predict the pretest probability of other disorders, including but not limited to, acute coronary syndrome and subarachnoid hemorrhage. The invention will also be applied to evaluate the probability of certain life-threatening diagnoses or other clinical outcomes in patients with symptoms or complaints, including, anterior chest pain, headache, syncope, symptoms consistent with transient ischemic attack, fever, minor head injury, shortness of breath, seizure, altered mental status, abdominal pain, trauma, dizziness, weakness, high blood pressure, and low blood pressure.

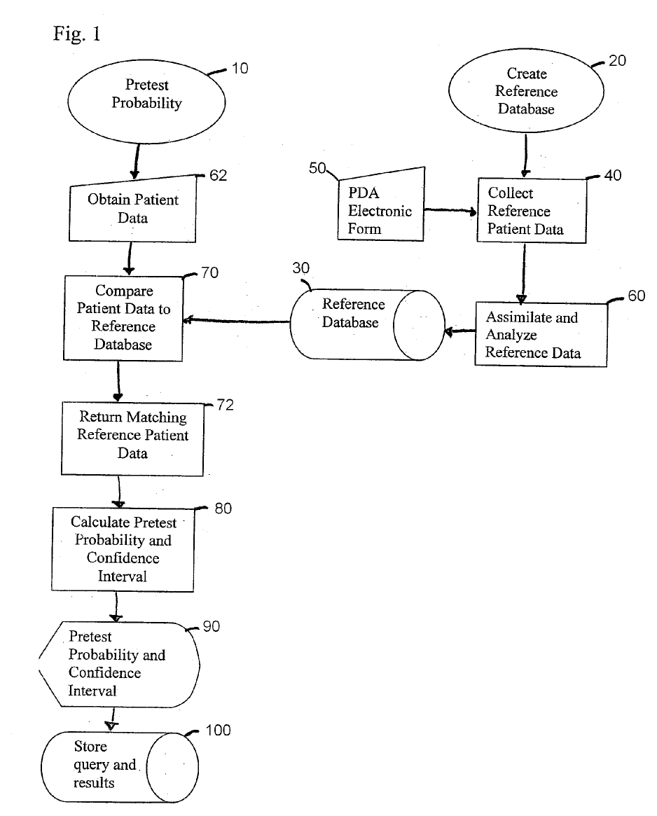

Referring now to the drawings in which like numerals refer to like parts through out, there is seen in FIG. 1 a flow chart of the method of present invention for determining a particular patient's pretest probability 10 for PE. Prior to starting the process for determining pretest probability 10 for a particular patient, however, it is necessary to create a reference database 20 containing clinical data for a sample population previously tested for PE which is then stored in reference database 30.

The collection of reference data 40 for reference database 30 may be performed by a variety of conventional methods, such as entering the information from a patient's medical history directly into a computer database. Alternatively, the present invention contemplates the use of an electronic form 50 programmed as part of an application for use, for example, on a personal digital assistance (PDA).

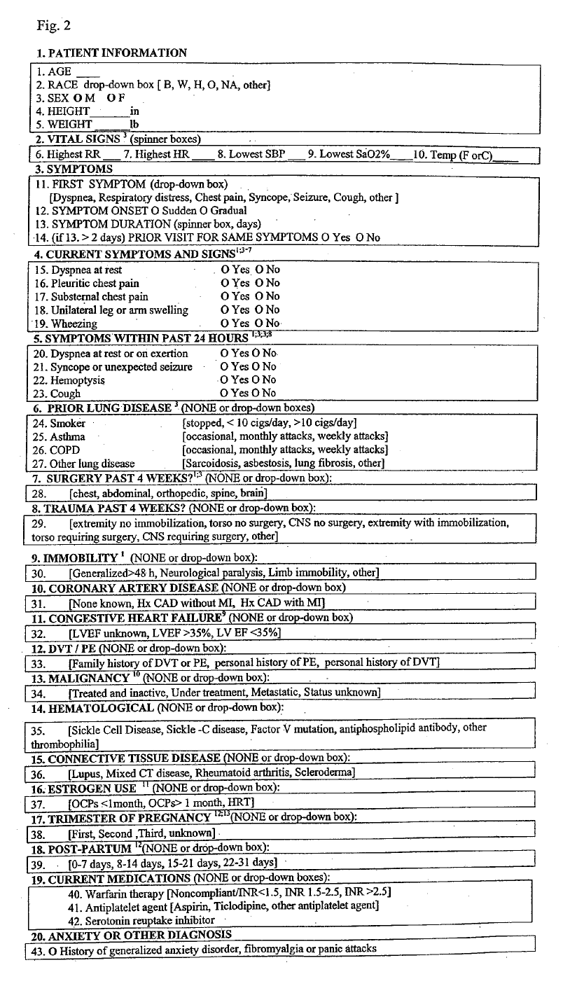

As seen in FIG. 2, electronic form 50 contains entry lines for a wide variety of information, from patient background information to specific clinical data relevant to diagnosis of a particular disease, such as a PE in the example case. A physician enters the answers to the numbered questions on electronic form 50 as they are obtained directly from the patient or from the patient's medical records.

Electronic form 50 is designed to allow very quick and easy input at the bedside. The content and number of fields in the form elicit data, which represent the pretest parameters that are immediately available at the bedside, including results of 12-lead electrocardiography. The prospective parameters that are included have previously been demonstrated to be important to the diagnosis and exclusion of PE based upon patient samples from emergency departments in the United States, Canada and Switzerland. Because electronic form 50 may be filled out in real-time in the PDA, its format allows the rapid entry of the key information that is most likely to help distinguish patients with PE from those without PE while minimizing the time requirement to enter the information.

After docking of the PDA containing electronic form 50 to a cradle device or other electronic means, newly created or updated electronic forms 50 are uploaded to a central computer or designated website programmed to assimilate and analyze the reference data 60. Electronic form 50 may alternatively be used to collect data from prospectively studied patients, retrospectively studied patients who were previously evaluated for PE, retrospectively studied patients with PE who were known to have been evaluated by a physician who failed to diagnose PE including patients who were the subject of civil litigation. It should be understood that the database can be built from as many sources as are required to provide sufficient reference data to establish a statistically significant database.

It is anticipated that a different database will be established to evaluate for the pretest probability of PE, acute coronary syndrome, subarachnoid hemorrhage and other life-threatening diseases. Separate databases will be assimilated to determine the probabilities of certain life-threatening diseases or outcomes for specific complaints, symptoms or signs including, anterior chest pain, headache, syncope, neurological symptoms consistent with transient brain ischemia, fever, minor head injury, shortness of breath, seizure, altered mental status, abdominal pain, trauma, dizziness, weakness, high blood pressure, and low blood pressure.

Once an accurate reference database 30 has been established, pretest probability 10 can be calculated from a personal computer or a personal digital assistant (PDA) which can access reference database 30 and which is programmed to perform a comparison of the patient data 70 to reference database 30. As seen in FIG. 1, patient data 62 is first obtained from the patient whose pretest probability 10 is to be determined. The data may comprise the same information which was obtained via electronic form 50 and used to compile the reference database, or may comprise only the most relevant, “cardinal” characteristics indicative of PE as determined by assimilating and analyzing the reference data 60. For example, multivariate logistic regression analysis or classification and regression tree analysis will be performed on reference database 30 to determine which parameters should be included as cardinal data to be used to estimate the probability of PE. Cardinal parameters may include continuous data, such as body mass index, age, gender, and vital signs as well as categorical data, such as gender, race, and the presence or absence of other factors.

Once patient data 60 is obtained, it is compared 70 to the reference database 30 to return matching reference patient data, i.e., reference patients with corresponding data points stored in reference database 30. Comparison 70 begins by taking the patient's individual data points, e.g., age, pulse rate, respiratory rate, systolic blood pressure, pulse oximetry, and temperature, and establishing a clinically relevant interval for each. Clinically relevant intervals are a given range for continuous data parameters. For example, patient age may be broken into 0-30 years, 31-45 years, 46-65 years, etc. The number and width of the intervals for each relevant parameter will be chosen based upon a histogram plot of the frequency (i.e., probability) of the disease versus the parameter. The width of the interval for each continuous parameter will be set to contain no more than 33.3% of the total number of patients in the database who are disease positive. The width of the interval will vary with the specific parameter, the size of the reference database, and the frequency of the disease in the reference database population. A match is determined by searching the database to see whether any reference patients in reference database 30 have a data point within the interval established for that parameter based upon data from the new patient for whom the pretest probability is unknown.

The cardinal parameters are expected to include, but not be limited to, symptoms (e.g., dyspnea, chest pain location, syncope, cough with hemoptysis, cough without hemoptysis), findings (e.g., unilateral leg swelling and wheezing on auscultation of the lungs), and risk factors (e.g., prior pulmonary embolism, recent surgery, malignancy, oral contraceptive use, pregnancy, and post partum status) and alternative processes (e.g., smoking, history of asthma, history of COPD, or other chronic lung disease). The patient is matched unconditionally to these cardinal parameters.

The patient may also be matched to additional data, termed “conditional” parameters, also recorded for each patient within reference database 30. Conditional parameters will have less importance in predicting the pretest probability according to the multivariate regression or classification and regression tree analysis. Conditional parameters significantly reduce the number of patients in the database that yield a match. As a result, pretest probability 10 can determined with and without conditional matches. A large disparity (e.g. >20%) between the pretest probability estimate using only cardinal parameters compared with the pretest probability obtained by matching of cardinal plus conditional parameters may indicate that the results of the former are not reliable. If in addition, the 95% confidence intervals for the pretest probability estimate from the cardinal plus conditional parameter match is very wide (e.g >30%), the results of the pretest probability estimate should be considered invalid for clinical decision-making. For the estimation of the pretest probability of PE, parameters that are likely to fall into the conditional category include, but are not limited to, duration of symptoms, the first symptom experienced, whether the patient has sought medical attention for the same complaint recently, body mass index, pregnancy, post-partum status, the presence or absence of sickle cell disease, connective tissue diseases, known coronary artery disease, congestive heart failure, family history of clots, estrogen replacement therapy, and history of anxiety or fibromyalgia.

By returning matching reference patients 72 using cardinal and conditional parameters, two pretest probabilities 80 with successively more exact matching and a decreasing number of matches can be determined. Thus, the application of method of the present invention will show the trade-off between precision of clinical matching and precision of the point estimate for pretest probability (based upon the 95% confidence interval). The first estimate will return the largest number of patients, matched for age and vital sign intervals, and exactly for cardinal features. The second will be all patients in the first group subjected to more exact matching for conditional variables.

Calculating the pretest probability and confidence interval 80 uses the traditional method to compute a 95% confidence interval. If x equals the number of subjects matched to a new patient and d equals the number of those matched subjects previously determined to have the disease in question, then the proportion of subjects with the disease (p), and the proportion of subjects without the disease (q) is calculated as follows:
p=d/x
q=1−p

The standard error [SE(p)] is determined according to the formula:
SE(p)√=(p*q)/√x

The formulas for calculating the upper and lower levels of the confidence intervals using a 95th percentile critical ratio from the normal distribution (1.96) are determined, respectively, as follows:
p+[1.96*SE(p)]
p−[1.96*SE(p)]

If d is less than 5 or if x-d is less than 5, then the “exact” methods as described by Newcomb and Altman, Chapter 6, Proportions and Their Differences in Statistics with Confidence, 2nd ed. (BMJ, Bristol, UK), hereby incorporated by reference, may be used.

Alternatively, using modification of the above formulae, other confidence intervals can be selected by the user, including a 99% confidence interval or the computation of the Bayesian credible interval. Once the pretest probability and associated confidence intervals are calculated 80, the results are displayed for the treating physician. Additionally, the query and results may be stored 110 along with date and time stamps to accurately record the entire pretest probability 10 process in permanent electronic storage.

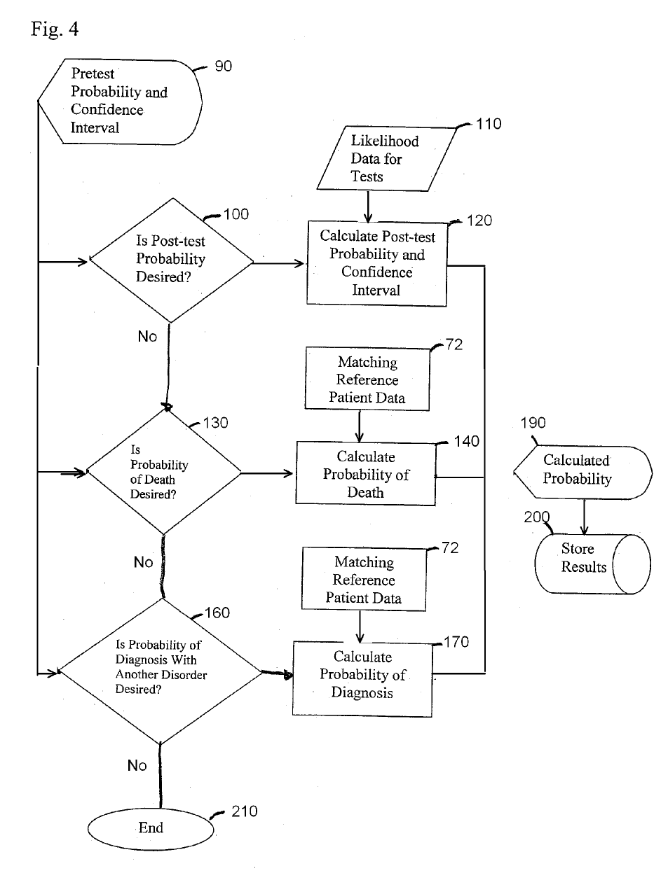

As seen in FIG. 4, reference database 30 can further be configured to return other important outcome data for use in calculating probabilities helpful to a physician. This data, in conjunction with calculated pretest probability 90, may be used to calculate post-test probabilities 120, the percentage of like patients who experienced death within 30 days of diagnosis 140, and the percentage of like patients who were ultimately diagnosed with another clinically important disease besides the disease under primary consideration 170.

For example, the returned results could include the number of patients in the matched set who were diagnosed with a myocardial infarction as opposed to PE.

For the purpose of calculating the post-test probability 120 of PE, the PDA or electronic device can be preprogrammed with published likelihood ratio data 110 for multiple tests, and the clinician can choose the test that he or she is considering. These tests include, but are not limited to, the D-dimer assay, contrast-enhanced computerized tomography angiography of the chest, scintillation ventilation-perfusion lung scanning (broken down into four results), echocardiography, normal plain film chest radiograph, normal alveolar deadspace measurement, and normal arterial oxygen partial pressure.

The post-test probability 120 (postP) of a disease is calculated by first obtaining the pre-test probability 90 (PreP) according to the present invention. The likelihood ratio negative (LRn) for a negative test result is calculated from published sensitivity and specificity data for the selected test according to the following formula:
LRn=(1−sensitivity)/specificity

The pre-test odds (PreO) and post-test odds (PostO) are then to be calculated as follows:
PreO=PreP/(1−PreP)
PostO=PreO*LRn

Finally, post-test probability 120 may be determined according to the following formula:
PostP=PostO/(PostO+1)

The probability of death may be calculated using the instances of death within the matching reference patient data 72. For example, the present invention can report the percentage of matched patients tested for PE who survived for three or more months without sequelae. If a physician desires, he or she can use matching reference patient 72 to determine the probability of adverse outcomes that would mandate specific treatment had the outcome been foreseen at the time of patient presentation. For example, for a patient with anterior chest pain, the present invention can report the percentage of matched patients from a chest pain reference database that required acute percutaneous coronary revascularization. The calculated probabilities for any or all of the these additional calculations 120, 140, 170 may be displayed 190 and stored in memory 200 with time and date stamps for future uploading to a server or an associated network storage device.

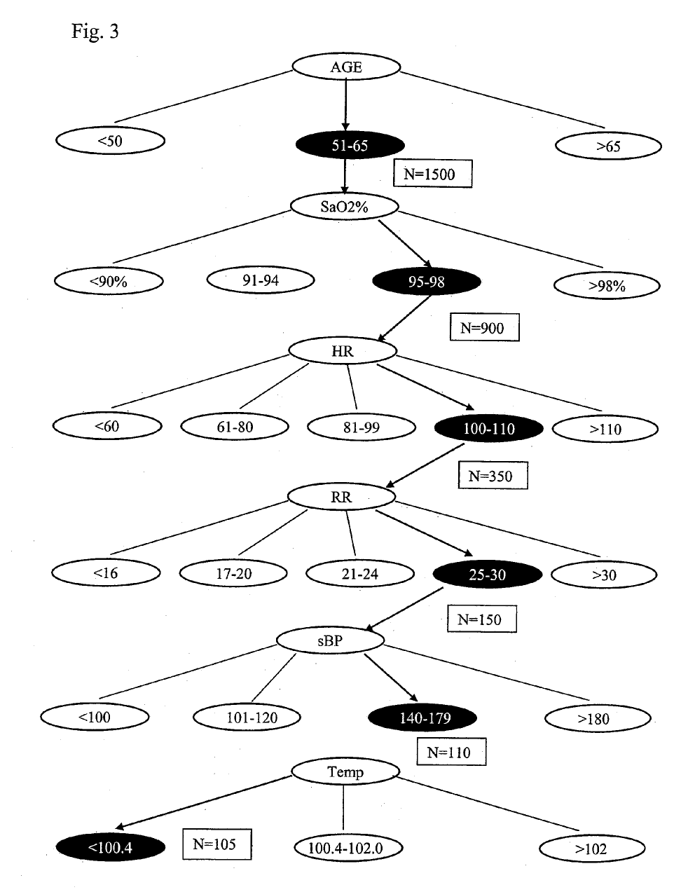

FIG. 3 depicts a clinical example of a linear comparison to a hypothetical reference database according to the method of the present invention for determining the probabilities associated with of a specific disease, PE. The patient in the example is a 57 year old white female with history of PE one year prior, presents with shortness of breath starting yesterday, nonproductive cough for one week and sudden onset pleuritic chest pain “exactly like” the previous PE last year. She smokes cigarettes and has been previously told she has the condition of fibromyalgia. She takes estrogen replacement therapy for hot flashes. Her vital signs are as follows: pulse 103, respiratory rate 28, sBP 141, SaO₂% 98%, and no leg swelling.

FIG. 3 also depicts the process of matching six successive continuous parameters from an unknown patient to the database, age, pulse oximetry (SaO₂%), heart rate (HR), respiratory rate (RR), systolic blood pressure (sBP), and temperature, (Temp). These parameters, their order, and the number and width of intervals for each parameter are shown for the purpose of describing the operation of the present invention and do not necessarily represent the order or criteria that will be used in actual practice.

As shown, the patient data is sorted and compared to the reference database, hypothetically containing 1,500 previously studied patients. The example patient is matched to patients in the database who also fall within the predetermined ranges shown (a match is represented by the darkened ovals). In this example, the process of matching the patient's age and vital signs has narrowed the number of patients in the hypothetical database down to 105 patients who have recorded clinical data similar to that of the sample patient.

The number of matches is further narrowed by matching considering the following data (with the hypothetical patient data in parenthesis): dyspnea (yes), syncope (no), substemal chest pain (no), pleuritic chest pain (yes), non-productive cough (yes), hemoptysis (no), oral contraceptives (no), prior PE or DVT (yes), active malignancy (no), recent Surgery (no), immobility (no), smoker (yes), asthma, COPD or other chronic lung disease (no), unilateral leg swelling (no), and wheezing (no). Consideration of these factors returns fifteen patients out of the 105 who had all of the cardinal parameters exactly the same as the patient's cardinal parameters. If one of the fifteen matches was ultimately diagnosed with PE, the example patient's pretest probability will be one-fifteenth or 6.7 percent, with a 95 percent confidence interval of zero to 32 percent, using the aforementioned formulae. Consideration of conditional variables narrows the number matches even further, returning a smaller set of patients (e.g., perhaps five) who are even more similar to the example patient.

The post-test probabilities of PE if the patient undergoes a CT scan of the chest, can be determined from the hypothetical results as follows. Assuming a likelihood ratio negative of 0.1 and likelihood ratio positive of 10, the post-test probability of PE after a negative CT scan would be 0.75% and the post-test probability of PE if the CT scan is positive would be 43%. The present invention may provide this computation for all other diagnostic tests with published likelihoods that are pertinent to the evaluation of the disease in question.

As seen in FIG. 4, the present system and method can also be used to perform attribute matching for assisting a physician in determining the degree of abnormality of a vital sign of a patient by evaluating a vital sign or signs against the vital sign of patients having similar attributes. Attribute matching provides a direct comparison of a patient of interest to those patients contained in a previously collected database who share the same clinical profile of the patient of interest. For example, a 23-year old white female who weighs 95 pounds and is 5′2″ tall, and has no disease co-morbidity, and no history of hypertension or hypotension, can be compared directly with patients of similar composition (e.g., white female age of greater than 18 but less than 30 years, with body weight between 90 and 100 pounds, height between 5 feet and 5′5″, and with no prior medical history). The system and method of the present invention will return only the females that match the attributes input into the profile, and their mean, standard deviation, range and other descriptive statistics of their systolic blood pressure. The results of the attribute matching of the present invention may be exported to a electronic medical record (EMR) system, printed onto hard copy for inclusion in the patient's chart, displayed in real time for evaluation by the physician or appropriate medical staff member, and/or stored in electronic format for future reference.

Assuming the blood pressure was measured in a setting similar to that of the patient of interest (e.g., emergency department, physician office, clinic or pharmacy), then the blood pressure of the patient of interest can be statistically compared to the results of the matched group. The elusive “standard in care” can thus help to be defined to determine whether the variable (in this case, systolic blood pressure) really was outside of expected ranges for a group of similar patients. This result would be useful in cases of risk management cases or medical malpractice cases.

Those of ordinary skill in the art would recognize that the same methodology could be used to determine multiple other dichotomous or ordinal variables. For example, to determine whether or not it is appropriate to disregard a family history of cancer when evaluating a patient with a history of blood in his/her stool. Without comparing a patient of concern to a large reference patient population, it is difficult to know the real significance of a family history of cancer without direct comparison to like patients. It is possible for logistic regression and other statistical methods to produce a measure of strengths of association between the factor (in this case, family history) and the outcome of interest in the individual patient.

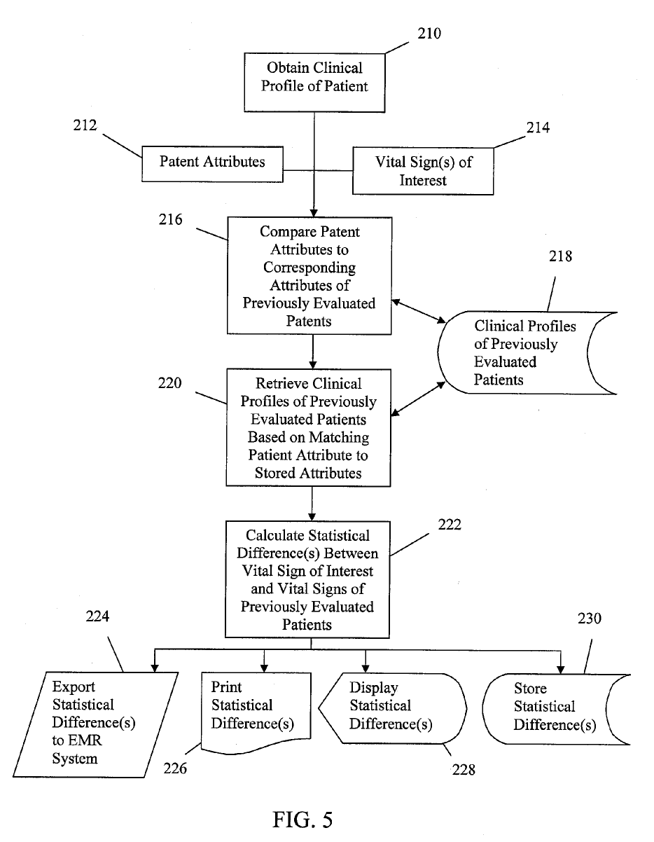

Referring to FIG. 5, the method of determining the degree of abnormality of a vital sign of a patient begins by obtaining the clinical profile of the patient 210. The clinical profile should include at least one patient attribute 212, such as height, weight, etc., and at least one vital sign of interest 214 whose degree of abnormality will be determined. The patient attribute(s) 212 are then compared 216 against previously evaluated patients whose clinical profiles have been stored in a database 218. Based on attribute matching of the patient attribute(s) 212 to the stored attributes 218, the clinical profiles of previously evaluated patients are retrieved 220. The statistical difference(s) are then calculated 222 based on a mathematical comparison between the patient's vital sign of interest 214 and the corresponding vital signs of the previously evaluated patients 218. The calculated statistical difference 222, as well as the patient attributes 212 and vital sign of interest 214 may then be exported to a electronic medical record (EMR) system 224, printed onto hard copy 226 for inclusion in the patient's chart, displayed in real time for evaluation by the physician or appropriate medical staff member 228, and/or stored in electronic format 230 for future reference.

What is claimed is:

1. The method of determining the degree of abnormality of at least one vital sign of a patient, comprising the steps of:
obtaining a clinical profile of said patient, wherein said clinical profile includes at least one patent attribute and at least one patient vital sign;
inputting said clinical profile into a data processing unit;
comparing said clinical profile of said patient to a database containing a plurality of stored clinical profiles of previously evaluated patients, wherein each clinical profile of each said previously evaluated patient includes at least one stored attribute corresponding to said at least one patent attribute and at least one stored vital sign corresponding to said at least one patient vital sign;
retrieving said clinical profiles of said previously evaluated patients from said database based on whether said stored attributes substantially match said at least one patient attribute;
calculating a statistical difference between said at least one patient vital sign and said stored vital signs of said previously evaluated patients.

2. The method of claim 1, further comprising the step of displaying said statistical difference.

3. The method of claim 1, further comprising the step of exporting said clinical profile of said patient and said statistical difference to an electronic medical record system.

4. The method of claim 1, further comprising the step of storing said clinical profile of said patient and said statistical difference in a computer storage medium.

5. The method of claim 1, further comprising the step of printing said clinical profile of said patient and said statistical difference.

6. A system for determining the degree of abnormality of at least one vital sign of a patient, comprising:
a data processing unit programmed to accept the inputting of a clinical profile for said patient, wherein said clinical profile includes at least one patent attribute and at least one patient vital sign;
a database containing a plurality of stored clinical profiles of previously evaluated patients in communication with said data processing unit, wherein each clinical profile of each said previously evaluated patient includes at least one stored attribute corresponding to said at least one patent attribute and at least one stored vital sign corresponding to said at least one patient vital sign;
wherein said data processing unit is further programmed to compare said clinical profile of said patient to said plurality of stored clinical profiles of previously evaluated patients and retrieve said clinical profiles of said previously evaluated patients from said database if said stored attributes substantially match said at least one patient attribute; and
wherein said data processing unit is programmed to calculate a statistical difference between said at least one patient vital sign and said stored vital signs of said previously evaluated patients.

7. The system of claim 6, further comprising a display in communication with said data processing unit for displaying said statistical difference.

8. The system of claim 6, wherein said data processing unit further comprises an interface for communicating with an electronic medical record system.

9. The system of claim 6, further comprising a non-volatile storage medium in communication with said data processing unit for storing said clinical profile of said patient and said statistical difference.

10. The system of claim 6, further comprising a printer in communication with said data processing unit for printing said clinical profile of said patient and said statistical difference.
